(19) United States
(12) Patent Application Publication
(10) Pub. No.: US 2026/0063616 A1
(43) Pub. Date: Mar. 5, 2026

(54) METHOD FOR QUANTITATIVELY DETECTING WATER CONTENT IN CONCRETE, DEVICE, MEDIUM, AND PRODUCT

(71) Applicant: Shenzhen University, Shenzhen City (CN)

(72) Inventors: Biqin Dong, Shenzhen City (CN); Shuxian Hong, Shenzhen City (CN); Xu Wang, Shenzhen City (CN); Yanshuai Wang, Shenzhen City (CN)

(21) Appl. No.: 19/053,655

(22) Filed: Feb. 14, 2025

(30) Foreign Application Priority Data
Aug. 28, 2024 (CN) 202411190762.2

(51) Int. Cl. G01N 33/38; G01N 23/04; G16C 60/00

(52) U.S. Cl. CPC G01N 33/38; G01N 23/04; G16C 60/00

(57) ABSTRACT

The present application provides a method for quantitatively detecting water content in concrete, a device, a medium, and a product, and relates to the technical field of detection for water content in concrete. The method includes: obtaining an X-ray image of a to-be-detected sample, where the to-be-detected sample is a to-be-detected concrete sample, and the X-ray image is an image obtained after an X-ray travels through the to-be-detected concrete sample; determining X-ray light field distribution information of the X-ray image according to the X-ray image; determining a water density of the to-be-detected sample according to the X-ray light field distribution information; and determining water content of the to-be-detected sample according to the water density. The present application can improve precision and efficiency of detection for water content in concrete, reduces detection costs, and is applicable to a large sample.

### CROSS REFERENCE TO RELATED APPLICATION

This patent application claims the benefit and priority of Chinese Patent Application No. 2024111907622, filed with the China National Intellectual Property Administration on Aug. 28, 2024, the disclosure of which is incorporated by reference herein in its entirety as part of the present application.

### TECHNICAL FIELD

The present application relates to the technical field of detection for water content in concrete, and in particular, relates to a method for quantitatively detecting water content in concrete, a device, a medium, and a product.

### BACKGROUND

Invasion of water into concrete is usually accompanied by diffusion of harmful ions. Therefore, detection for water distribution in concrete is of great significance to research of concrete durability and safety, and engineering protection. The existing detection methods mainly include a gravimetric method, an electrical resistance method, an electrical conductivity method, a neutron radiography method, a nuclear magnetic resonance method, and the like. The gravimetric method is the original measurement method in which a water content change in a sample is determined through weighing. However, in the gravimetric method, only total water content in the sample can be roughly estimated, and local water content information cannot be provided. Moreover, when the sample is large, it is usually difficult to meet weighing conditions. The electrical resistance method and the electrical conductivity method are methods for estimating water content in a sample by using electrical parameters. Fast two-dimensional imaging of water content distribution can be provided by the two methods, but measurement results of the two methods are usually affected by a plurality of factors, such as distribution of reinforcing steel bars in the concrete, chloride ion content, and an ambient temperature. Therefore, accuracy of the measurement results of the electrical resistance method and the electrical conductivity method is not high. Although high-resolution and high-sensitivity detection results can be provided by the neutron radiography method and the nuclear magnetic resonance method, the two methods are only applicable to detection of a small sample, and have limitations. For example, the nuclear magnetic resonance method is limited by components of the sample and is currently applicable to only white cement. The neutron radiography method is too expensive and is applicable to only a detection scenario in a laboratory.

In conclusion, none of the existing methods for detecting water content in concrete has the advantages of a high speed, high precision, low costs, applicability to a large sample, and the like at the same time. Therefore, how to provide a method for quantitatively detecting water content in concrete with a high speed, high precision, low costs, and applicability to a large sample has become an urgent technical problem to be resolved in this field.

### SUMMARY

An objective of the present application is to provide a method for quantitatively detecting water content in concrete, a device, a medium, and a product, to improve precision and efficiency for detecting water content in concrete, reduce detection costs, and achieve applicability to a large sample.

To achieve the above objective, the present application provides the following technical solutions.

According to a first aspect, the present application provides a method for quantitatively detecting water content in concrete. The method for quantitatively detecting water content in concrete includes:
obtaining an X-ray image of a to-be-detected sample, where the to-be-detected sample is a to-be-detected concrete sample, and the X-ray image is an image obtained after an X-ray travels through the to-be-detected concrete sample;
determining X-ray light field distribution information of the X-ray image according to the X-ray image;
determining a water density of the to-be-detected sample according to the X-ray light field distribution information; and
determining water content of the to-be-detected sample according to the water density.

According to a second aspect, the present application provides a computer device, including a memory, a processor, and a computer program stored in the memory and executable on the processor, where the processor executes the computer program to perform the steps of the method for quantitatively detecting water content in concrete in the first aspect.

According to a third aspect, the present application provides a computer storage medium. The computer storage medium stores a computer program, where the program is executed by a processor to implement the method for quantitatively detecting water content in concrete in the first aspect.

According to a fourth aspect, the present application provides a computer program product, including a computer program, where when executed by a processor, the computer program implements the method for quantitatively detecting water content in concrete in the first aspect.

According to specific examples provided in this application, this application discloses the following technical effects.

According to the method for quantitatively detecting water content in concrete, the device, the medium, and the product provided in the present application, the water density and the water content can be sequentially obtained according to the X-ray light field distribution information by only collecting the X-ray image of the to-be-detected sample and obtaining the X-ray light field distribution information, such that quantitative detection for water content in a concrete sample is implemented. In the present application, the X-ray image of the concrete sample only needs to be shot, and an ultra-large detection device does not need to be used, such that detection is simpler, and detection costs are lower. In addition, electrical parameters such as a resistivity and a conductivity are not involved, and therefore, a calculation process is simple. Total water content inside the sample is not roughly estimated through weighing, and water content in the concrete is objectively and scientifically analyzed based on the X-ray image and the X-ray light field distribution information thereof. Therefore, the method is higher in accuracy, is applicable to detection for water content in various large concrete samples, and effectively reduces detection costs on the premise of improving precision and efficiency of detection for water content in concrete.

### BRIEF DESCRIPTION OF THE DRAWINGS

To describe the technical solutions in the examples of this application or in the prior art more clearly, the following briefly describes the accompanying drawings required for the examples. Apparently, the accompanying drawings in the following description show merely some examples of this application, and a person of ordinary skill in the art may still derive other accompanying drawings from these accompanying drawings without creative efforts.

FIG. 1 is a flowchart of a method for quantitatively detecting water content in concrete according to an embodiment of the present application;

FIG. 2 is a schematic diagram of a structure of an X-ray source according to an embodiment of the present application;

FIG. 3 is a schematic diagram of a structure of an X-ray emitting apparatus according to an embodiment of the present application;

FIG. 4 is a schematic diagram of a layout manner of an X-ray detection system according to an embodiment of the present application;

FIG. 5 is a schematic diagram of a principle at a calibration stage and a detection stage according to an embodiment of the present application;

FIG. 6 is a front view of a calibration model according to an embodiment of the present application;

FIG. 7 is a stereoscopic view of a calibration model according to an embodiment of the present application;

FIG. 8 is a schematic diagram of a shooting manner of a calibration model according to an embodiment of the present application; and

FIG. 9 is a schematic diagram of a structure of a computer device according to an embodiment of the present application.

### DETAILED DESCRIPTION OF THE EMBODIMENTS

The technical solutions in the embodiments of the present application are clearly and completely described below with reference to the drawings in the embodiments of the present application. Apparently, the described embodiments are only some rather than all of the embodiments of the present application. All other embodiments obtained by a person of ordinary skill in the art based on the embodiments of the present application without creative efforts shall fall within the protection scope of the present application.

To make the above objectives, features, and advantages of the present application more obvious and easier to understand, the present application will be further described in detail with reference to the accompanying drawings and specific implementations.

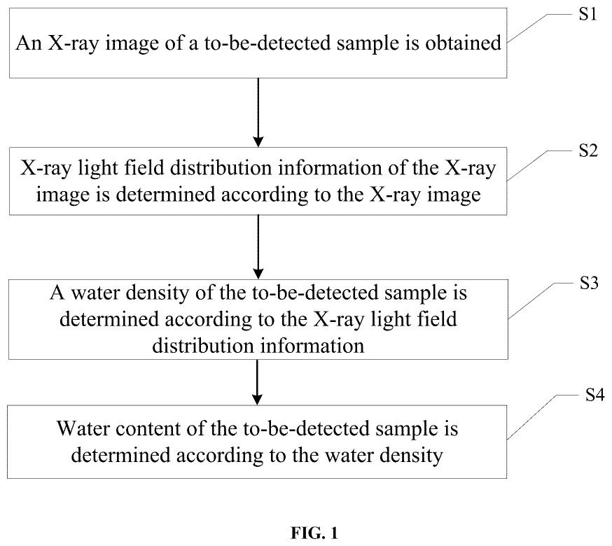

As shown in FIG. 1, an embodiment provides a method for quantitatively detecting water content in concrete. The method for quantitatively detecting water content in concrete is mainly used to measure a value of water content in concrete, and specifically includes the following steps.

In step S1, an X-ray image of a to-be-detected sample is obtained, where the to-be-detected sample is a to-be-detected concrete sample, and the X-ray image is an image obtained after an X-ray travels through a to-be-detected concrete sample.

In step S2, X-ray light field distribution information of the X-ray image is determined according to the X-ray image.

In step S3, a water density of the to-be-detected sample is determined according to the X-ray light field distribution information.

In step S4, water content of the to-be-detected sample is determined according to the water density.

In this embodiment, the step S1 of obtaining an X-ray image of a to-be-detected sample specifically includes the following step:

The to-be-detected sample is detected by an X-ray detection system to obtain the X-ray image.

In this embodiment, the X-ray detection system at least includes an X-ray emitting apparatus and an X-ray detector, and further includes an image processing apparatus, and the like. Both the X-ray emitting apparatus and the X-ray detector are connected to the image processing apparatus, the X-ray emitting apparatus and the X-ray detector are disposed opposite to each other, and the to-be-detected sample is placed between the X-ray emitting apparatus and the X-ray detector. The X-ray emitting apparatus is configured to emit an X-ray to the to-be-detected sample. The X-ray detector is configured to: receive the X-ray that passes through the to-be-detected sample, convert X-ray energy into a recordable electric signal, and send the electric signal to the image processing apparatus. In this embodiment, the image processing apparatus may be a terminal like a computer, a notebook computer, a tablet computer, and a cloud server. In this embodiment, the image processing apparatus is preferably the computer, and is mainly configured to generate, according to the electric signal sent by the X-ray detector, the X-ray image of the to-be-detected sample by combining an X-ray emission status of the X-ray emitting apparatus, and is further configured to perform data analyzing and processing on the X-ray image, for example, obtaining greyscale information of the X-ray image, obtaining the X-ray light field distribution information, and calculating data such as the water density and water content.

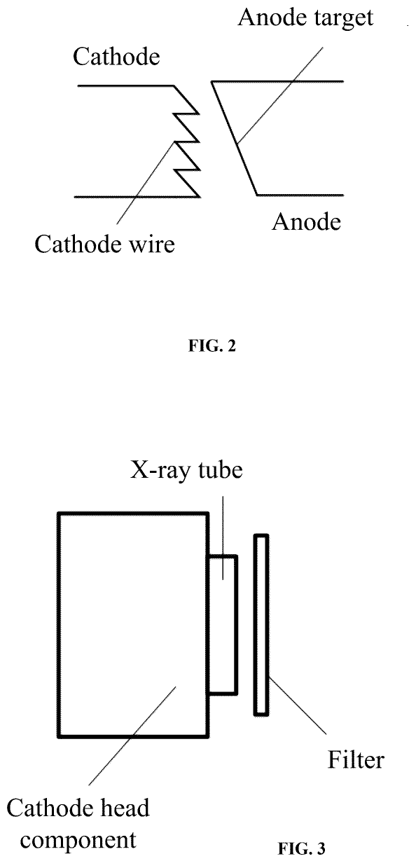

In this embodiment, the X-ray emitting apparatus mainly includes structures such as an X-ray source, and a hardware filter component. The X-ray source is responsible for emitting an X-ray. As shown in FIG. 2, a high voltage of up to tens of kilovolts is applied to both ends of a cathode wire of the X-ray source, such that an anode target is bombarded after kinetic energy is obtained by electrons on the cathode wire. In this way, the X-ray is emitted by the anode target from an X-ray tube. In this embodiment, in view of a working principle of the X-ray tube, the X-ray emitted by the X-ray tube is polychromatic light. Fort the polychromatic light, a wavelength of the X-ray has a non-zero value within a large spectral range, such that there are high-energy photons and low-energy photons in the emitted X-ray. Therefore, the hardware filter component in this embodiment is a filter. The filter is disposed in front of an emission window of the X-ray tube. As shown in FIG. 3, the filter may be a metal plate containing heavy metal. A low-energy X-ray is attenuated easier than a high-energy X-ray in a transmission process, and therefore, a proportion of low-energy X-ray can be reduced by the filter in this embodiment. In this embodiment, there is a pore between the cathode wire and the anode target. Both the cathode wire and the anode target are disposed in the X-ray tube, cannot be separated, and are of an independent integrated structure. The X-ray tube is connected to a cathode head component. The cathode head component is configured to provide a high voltage for the X-ray tube. The filter is generally independent of the X-ray source, and is independently disposed in a slot in front of the X-ray source.

In this embodiment, the X-ray detector is an X-ray detector array including a plurality of detectors. The detectors are configured to: collect and record the X-ray image obtained after the X-ray emitted by the X-ray emitting apparatus travels through the to-be-detected sample. The X-ray image is usually a greyscale image, and is configured to analyze a water content distribution status inside the sample. Different space angles are formed among the X-ray emitting apparatus, the X-ray detector, and the to-be-detected sample. Therefore, when passing through the to-be-detected sample, the X-ray is attenuated to different extents according to the Lambert-Beer law, and a light intensity I of the attenuated X-ray may be represented as follows:

$$I = {I}_{0} {e}^{μ t} \qquad ( 1 )$$

where, I₀ is an initial X-ray light intensity, μ is an attenuation coefficient, t is a thickness of the to-be-detected sample, and e is a natural constant. The X-ray is transmitted inside the sample at different angles, and therefore, the thickness t of the to-be-detected sample varies with the angle. A distribution condition of different X-ray intensities detected by the plurality of detectors is analyzed to obtain an actual distribution condition of thicknesses inside the sample. It should be noted that, the thickness herein is a physical conception rather than an actual spatial size of the to-be-detected sample, and does not refer to a thickness of the to-be-detected sample. The thickness of the to-be-detected sample and the equivalent thickness of the water contained in the to-be-detected sample are respectively represented as a and b. However, distribution of water content inside the sample is uneven, and there is no varying spatial distance size. Therefore, the thickness in this embodiment is a hypothetical physical model for convenience.

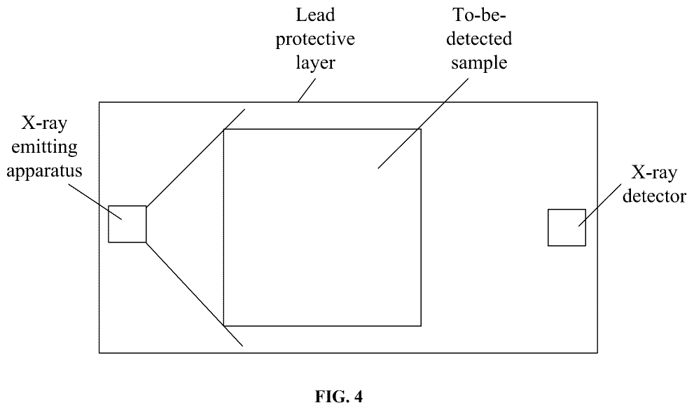

As shown in FIG. 4, in this embodiment, when the X-ray detection system is configured to detect, a sample placement area is disposed between the X-ray emitting apparatus and the X-ray detector. The X-ray placing area is configured to place a to-be-detected concrete sample, and can be configured to place a large concrete sample or a small concrete sample according to a need. A lead protective layer is further disposed at a periphery of the X-ray detection system, such that radiation is prevented through safety protection measure of the lead protective layer, and body safety in a measurement process is ensured. During actual use, the X-ray emitting apparatus in the X-ray detection system is configured to emit an X-ray of a specific light intensity. The X-ray is attenuated after being transmitted through the to-be-detected sample to form unique light field distribution with water content information behind the to-be-detected sample, and is received by the X-ray detector. The X-ray detector is configured to: collect the X-ray image including light field distribution information and transmit the X-ray image to the image processing apparatus. The image processing apparatus is configured to obtain water content information of the to-be-detected concrete sample through image analyzing and processing and data calculating.

In this embodiment, the step S2 of determining X-ray light field distribution information of the X-ray image according to the X-ray image specifically includes the following steps.

In step S21, image processing is performed according to the X-ray image to determine greyscale values corresponding to different positions of the X-ray image.

In step S22, the X-ray light field distribution information is determined according to the greyscale values corresponding to the different positions of the X-ray image.

In this embodiment, the step S3 of determining a water density of the to-be-detected sample according to the X-ray light field distribution information specifically includes the following steps.

In step S31, the to-be-detected sample is calibrated to obtain a calibration result, where the calibration result includes an attenuation coefficient curve formula and a parameter value thereof, and a water absorption function formula and a parameter value thereof. The attenuation coefficient curve formula and the parameter value thereof are mainly used for subsequent calculation of the water density of the to-be-detected sample. The water absorption function formula and the parameter value thereof are mainly used for subsequent calculation of the water content of the to-be-detected sample.

In step S32, the water density of the to-be-detected sample is calculated by substituting the X-ray light field distribution information into the attenuation coefficient curve formula.

In this embodiment, the step S31 of calibrating the to-be-detected sample to obtain a calibration result specifically includes the following steps.

In step S311, basic information of the to-be-detected sample is obtained, where the basic information includes a density, a cement-to-water ratio, a cement type, a cement-to-sand ratio, gradation, and an admixture type and proportion.

In step S312, a calibration model with a composition the same as that of the to-be-detected sample is prepared according to the basic information of the to-be-detected sample, where the calibration model includes a first calibration model and a second calibration model, and the first calibration model is the same as the second calibration model.

In step S313, vacuum water-saturation is performed on the first calibration model, the first calibration model is dried to constant weight, the second calibration model is soaked in deionized water until the second calibration model is completely saturated, the first calibration model and the second calibration model are respectively weighed, and mass of the first calibration model and mass of the second calibration model as well as a mass difference between the first calibration model and the second calibration model are determined.

In step S314, a substrate surface of the first calibration model is fitted to a substrate surface of the second calibration model, such that a side surface of the first calibration model and a side surface of the second calibration model are perpendicular to each other to obtain an integrally spliced model.

In step S315, an X-ray image of the integrally spliced model is obtained, and X-ray light field distribution information at each position of the integrally spliced model is determined according to the X-ray image.

In step S316, each parameter value of the attenuation coefficient curve formula and each parameter value of the water absorption function formula are determined according to the X-ray light field distribution information at each position of the integrally spliced model.

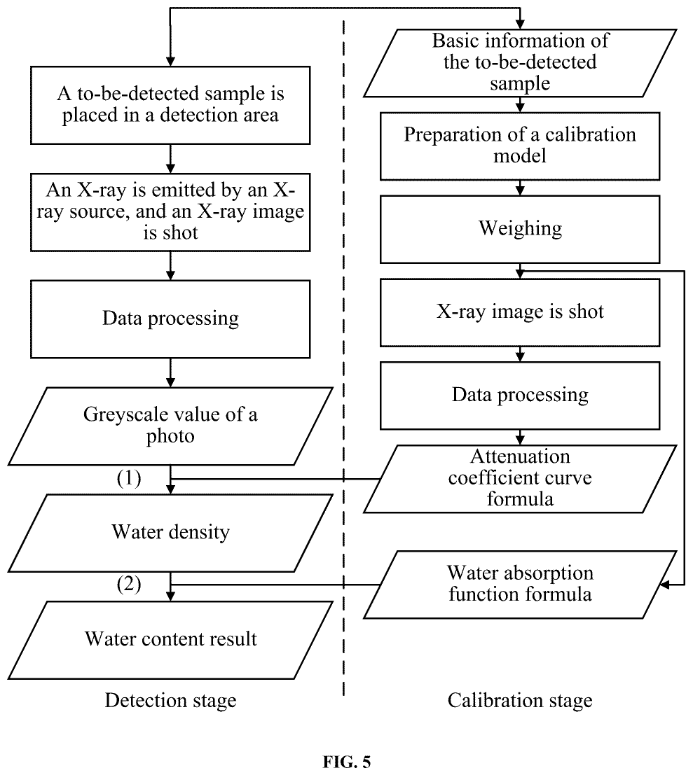

It should be noted that, the step of calibrating the to-be-detected sample may be completed before the X-ray image of the to-be-detected sample is collected, or may be completed before the X-ray light field distribution information is determined according to the X-ray image at an image processing stage. The to-be-detected sample is mainly calibrated to determine the attenuation coefficient curve formula and the water absorption function formula. The attenuation coefficient curve formula and the water absorption function formula are respectively provided in subsequent calculation processes in step S3 and step S4, as shown in FIG. 5. Therefore, the to-be-detected sample only needs to be calibrated before the water density of the to-be-detected sample is calculated.

In this embodiment, the step S4 of determining water content of the to-be-detected sample according to the water density specifically includes the following step.

The water density is substituted into the water absorption function formula to obtain the water content of the to-be-detected sample through calculation.

In this embodiment, the detection process for water content in concrete may be divided into two stages: a calibration stage and a detection stage. Both the calibration stage and the detection stage are implemented through a working manner of a hardware device of the X-ray detection system.

As shown in FIG. 5, the calibration stage is shown on the right side of a dashed line in FIG. 5, and the detection stage of the to-be-detected sample is shown on the left side of the dashed line. In this embodiment, calibration is performed to obtain a necessary calibration result. The calibration result includes two parts: the attenuation coefficient curve formula (denoted as (1) in FIG. 5), and the water absorption function formula (denoted as (2) in FIG. 5). In the detection process of the to-be-detected sample, the water density can be obtained by substituting specific X-ray light field distribution into the attenuation coefficient curve formula, and the water content can be obtained by substituting the water density into the water absorption function formula.

In this embodiment, a step-shaped concrete sample is used as an example. For example, the step-shaped concrete sample is used as a calibration model for describing a specific calibration process. The specific calibration process includes the following steps.

In step (1), basic information of the to-be-detected sample is obtained, where the basic information includes a density, a cement-to-water ratio, a cement type, a cement-to-sand ratio, gradation, and an admixture type and proportion. A typical density of the concrete sample is 1.8 kg/m³ to 3 kg/m³. The cement-to-water ratio is a ratio of cement to water, and is usually between 0.3 and 0.5. A common cement type includes silicate cement, aluminate cement, sulphoaluminate cement, fluoraluminate cement, ferroaluminate cement, and the like. There may also be special cement. These cement types are factors that affect a mechanical property, a porosity, a density and the like of the sample, but do not affect applicability of the technical solution in this embodiment. The cement-to-sand ratio is a ratio of cement to used sand, and is usually between 0 and 4, where sand is not used in all samples. Gradation refers to grain size gradation of coarse aggregates, both sand (fine aggregates) and coarse aggregates (gravels) are used in concrete. Different grain sizes are used in different buildings. For example, only 5-20 mm gravels (with a maximum grain size not exceeding 20 mm) are used for primarily-graded concrete. Both 5-20 mm gravels and 20-40 mm gravels (with a maximum grain size not exceeding 40 mm) are used for secondarily-graded concrete. 5-20 mm gravels, 20-40 mm gravels, and 40-80 mm gravels (with a maximum grain size not exceeding 80 mm) are used for three-graded concrete. The admixture type is another admixture rather than cement, water, sand and coarse aggregates in the concrete, and includes silica fume, fly ash, slag, and the like of waste in industrial production. For saving costs, these admixtures may be mixed into the cement for being stirred, and poured (however, this type of mixture is not in all concrete).

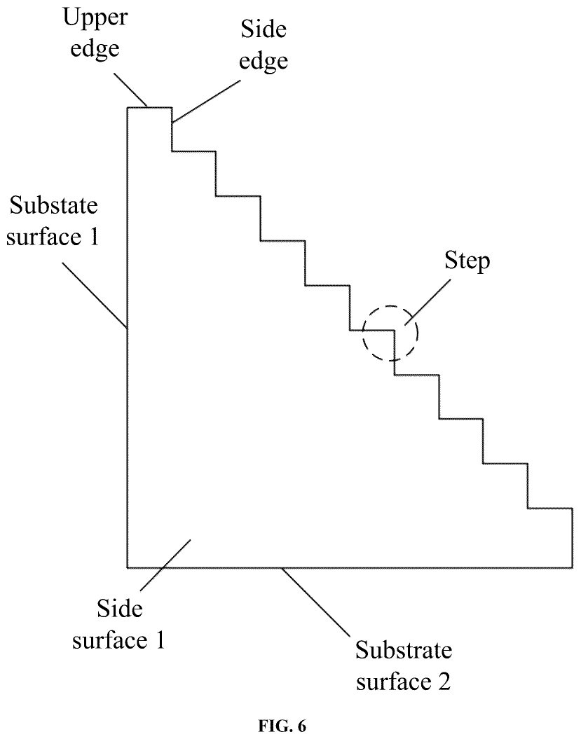

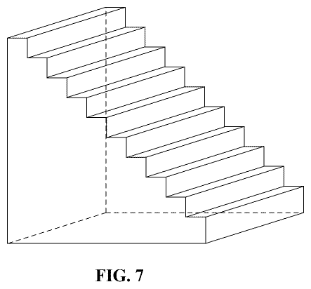

In step (2), two calibration models with composition the same as that of the to-be-detected sample are prepared, where shapes of the two calibration models are as shown in FIG. 6 and FIG. 7. The calibration model is step-shaped, and includes two substrate surfaces, two side surfaces, and several steps, where each step includes an upper edge and a lower edge. The substrate surface is a flat square plane, and is molded by an edge of a mold during sample pouring. The side surface of the calibration model is a flat plane, and is parallel to an incident direction of the X-ray during measurement. The step is of a right-angled structure in which the upper edge and the lower edge are perpendicular to each other. There are usually three or more steps. The calibration result is not affected by a quantity of steps. When the quantity of steps is large, the step-shaped calibration model is approximately in a shape of triangular prism, as shown in FIG. 6. In this case, the calibration result is not affected either. In this embodiment, a calibration model with three steps is preferred. When the calibration model is prepared, cement and water are mixed and stirred to form a flowable mixture, and therefore, the flowable mixture needs to be poured into the mold and only can be solidified after a period of time. In this case, the mold is removed to form the calibration model that is completely made of hardened cement. Therefore, form stripping and maintenance are performed on the two calibration models according to an actual condition of the to-be-detected sample. After being maintained, the two calibration models are respectively operated as follows: Vacuum water-saturation is performed on one calibration model, and then the calibration model is dried to constant weight, where the calibration model is denoted as a first calibration model. The other calibration model is soaked into deionized water until the calibration model is statured, and the calibration model is denoted as a second calibration model. After being prepared, the first calibration model and the second calibration model are weighed, and mass m₁ of the first calibration model, mass m₂ of the second calibration model, and a mass difference Δm between the first calibration model and the second calibration model are recorded. A physical meaning of Δm is mass of free water in the second calibration model. The first calibration model is completely dry, and the second calibration model is in a water-saturated state after being soaked. Therefore, a difference between the mass m₁ of the first calibration model and the mass m₂ of the second calibration model is a value of complete water content in the second calibration model, namely, Δm. In this embodiment, the calibration model includes a water-free calibration model and a watery calibration model. The water-free calibration model is the first calibration model, and the watery calibration model is the second calibration model.

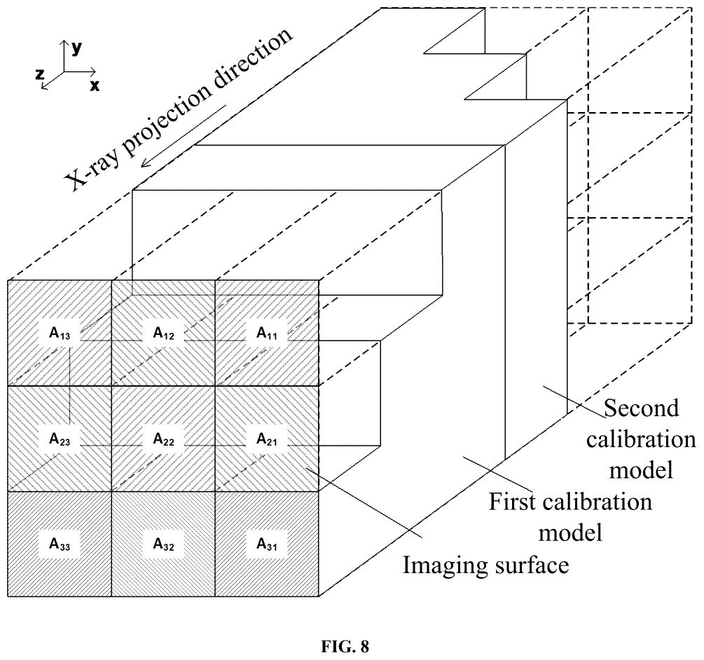

In step (3), a substrate surface of the first calibration model is fitted to a substrate surface of the second calibration model. In this case, the two selected substrate surfaces are referred to as fitted substrate surfaces, and side surfaces of the first calibration model and the second calibration model should be ensured to be perpendicular to each other. A placement model of the first calibration model and the second calibration model is as shown in FIG. 8. As shown in FIG. 8, both the first calibration model and the second calibration model are calibration models each having 3 steps. The calibration result is not affected by the quantity of steps. Therefore, a calibration model having 4 steps, 5 steps, 6 steps, or the like may alternatively be selected. In this embodiment, two calibration models each having 3 steps are selected for convenience in describing and explaining a principle. It is easy to understood that a spatial position relationship and a calibration result of the two calibration models are also effective in a calibration model having more steps. Due to different distances from steps to fitted substrate surfaces, lengths for the X-ray to pass through the first calibration model and the second calibration model are different. Due to a difference between water content in the first calibration model and water content in the second calibration model, the difference between lengths further includes the difference in water content. The difference is finally reflected as an intensity distribution difference obtained after the X-ray is attenuated through the first calibration model and the second calibration model. The light field intensity difference is also marked in an Aₘₙ form on an imaging surface in FIG. 8. For example, A₁₂ in FIG. 8 is a light filed obtained after the X-ray is attenuated through a first step of the first calibration model and a second step of the second calibration model. In addition, A₂₁ in FIG. 8 is a light filed obtained after the X-ray is attenuated through a second step of the first calibration model and a first step of the second calibration model. For A₁₂ and A₂₁, distances for the X-ray to pass through the first calibration model and the second calibration model are the same. However, the X-ray is additionally attenuated in A₁₂ by water contained in the second calibration model, and therefore, a light field intensity of the X-ray is weak. Light field intensities in areas such as A₁₁ to A₃₃ are different.

In this embodiment, after the light field distribution difference is obtained, the light intensities can be represented as I(a, b), where a represents a distance that the X-ray passes through the water-free first calibration model, and b represents a distance that the X-ray passes through the watery second calibration model. Different Aₘₙ have different values of a and b. In this embodiment, the water-free calibration model is the first calibration model, and the watery calibration model is the second calibration model. Different I(a, b) corresponding to different areas are substituted into formula (2):

$$- ln ( \frac{I ( a , b )}{{I}_{0}} ) = \frac{{( {k}_{1} a + {k}_{2} b )}^{2} + {k}_{3} a + {k}_{4} b + {k}_{5}}{{k}_{6} a + {k}_{7} b + {k}_{8}} \qquad ( 2 )$$

where, I₀ is an initial X-ray light intensity, and k₁, k₂, k₃, k₄, k₅, k₆, k₇, and k₈ are parameters to be solved. Each of the two calibration models has at least three steps, and therefore, there are at least 9 different I(a, b) of a and b. In this embodiment, each of the first calibration model and the second calibration model has three steps, and therefore, 9 different I(a, b) of a and b are obtained. In formula (2), there are only k₁ to k₈, 8 parameters to be resolved in total. Therefore, all the parameters to be solved in formula (2) can be solved. Formula (2) is the attenuation coefficient curve formula that describes a mathematical relation between a light intensity attenuation degree and water content. In addition, a total value of water content in the second calibration model is Δm, and formula (3) can be obtained as follows:

$$Δ m = \sum \frac{Δ wb}{a + b} . \qquad ( 3 )$$

In formula (3), Δm represents the water content, namely, water content in the to-be-detected sample, that is, water absorption of the to-be-detected sample, Δw represents a water absorption rate, a represents a distance that the X-ray passes through the water-free first calibration model, and b represents a distance that the X-ray passes through the watery second calibration model.

In this embodiment, the water content is a known value, and water content in each grid in the second calibration model is required. Water content and a greyscale change value are provided in each cell. The water content is obtained according to formula (3), and the greyscale change value is obtained through the X-ray image.

In formula (3), the water absorption rate Δw can be represented as follows:

$$Δ w = \frac{{m}_{2} - {m}_{1}}{{m}_{1}} . \qquad ( 4 )$$

In formula (4), Δw represents the water absorption rate, m₁ represents mass of the first calibration model, and m₂ represents mass of the second calibration model.

In the calibration process, both a and b are variables. In an actual measurement process, a distance a for the X-ray to pass through the water-free first calibration model in formula (2) is actually a thickness of the to-be-detected sample. The thickness a of the to-be-detected sample is known, and therefore, there is actually only one independent variable in formula (2), represented as an

$$ln ( \frac{I ( a , b )}{{I}_{0}} )$$

value changing with the water content b, namely, a curve, referred to as an attenuation coefficient curve. The water content b is inversely derived from

$$ln ( \frac{I ( a , b )}{{I}_{0}} ) ,$$

such that the water content is finally calculated. The relation between the light intensity attenuation degree and the water content can be converted into a proportional value of Δm₀. Formula (3) is the water absorption function formula. In this way, the calibration process is completed.

In this embodiment, after the attenuation coefficient curve is obtained, the calibration process is completed. The entire measurement process is follows: First, water content is provided by the first calibration model and the second calibration model (water content corresponding to each step of Amn). Then, a greyscale value, namely,

$$ln ( \frac{I ( a , b )}{{I}_{0}} )$$

is provided by the X-ray image of the first calibration model and the second calibration model. Then, water content information is combined with greyscale value information to obtain the attenuation coefficient curve formula. Then, any to-be-detected sample is measured, and the X-ray image of the to-be-detected sample is collected to obtain the greyscale value information. Finally, the water content is inversely derived from the greyscale value information.

In this embodiment, the measurement process of the to-be-detected sample can be implemented after the attenuation coefficient curve formula and a corresponding parameter thereof and the water absorption function formula and a corresponding parameter thereof are obtained through the calibration method. The to-be-detected sample is placed in the X-ray detection system according to the method shown in FIG. 4, an X-ray is emitted to the to-be-detected sample through the X-ray emitting apparatus, and the X-ray image is obtained by the X-ray detector through shooting. The X-ray image is usually a greyscale image, and therefore, the water density and the water content can be obtained by sequentially substituting the greyscale value change in the X-ray image into the attenuation coefficient curve formula and the water absorption function formula. For example, it is assumed that the X-ray is not incident to the to-be-detected sample, an initial X-ray intensity is I₀. After the X-ray is incident to the to-be-detected sample, distribution of the X-ray intensities inside the to-be-detected sample is I(x, y). In this case, an attenuation coefficient at each point inside the to-be-detected sample is ln I/I₀, where (x, y) in I(x, y) are coordinates of each point of internal space of the to-be-detected sample, that is, attenuation coefficients at points inside the to-be-detected sample are different. For a to-be-detected sample with a thickness, an attenuation coefficient of the to-be-detected sample is in one-to-one correspondence with water content. A two-dimensional distribution result of the water content in the sample can be obtained by substituting the attenuation coefficient into the attenuation coefficient curve formula and further substituting a result obtained according to the attenuation coefficient curve formula into the water absorption function formula.

In this embodiment, based on the two calibration results: the attenuation coefficient curve and the water absorption function, calibration work is performed before the measurement process, that is, the calibration stage is previous to the detection stage, such that real-time performance of the measurement process is ensured, time for each measurement is within one minute, a detection speed is increased, and detection efficiency is improved. In another aspect, the X-ray image is analyzed and processed in this application, and the water content in concrete is calculated with reference to the attenuation coefficient curve and the water absorption function, such that accuracy of quantitative detection for water content is effectively improved, and a measurement error is less than 5%, while an error calibrated for water content in traditional X-ray imaging is 26.9%. The detection method in this embodiment has a higher detection speed and higher accuracy.

In this embodiment, an X-ray absorption imaging method is used to implement quantitative detection on water content in the concrete. After the X-ray travels through the sample, an intensity of the X-ray is attenuated due to photoelectric effect and Compton scattering. Based on the working principle of the X-ray tube, the X-ray is polychromatic light, and therefore, X-ray hardening effect is generated in an imaging process. X-ray hardening is corrected through an X-ray hardening correction method, to implement quantitative detection for water content in the concrete. The quantitative detection integrates a plurality of advantages such as a high speed, low costs, a high accuracy rate, high sensitivity, a simple structure, no contact, nondestructive measurement, and two-dimensional imaging.

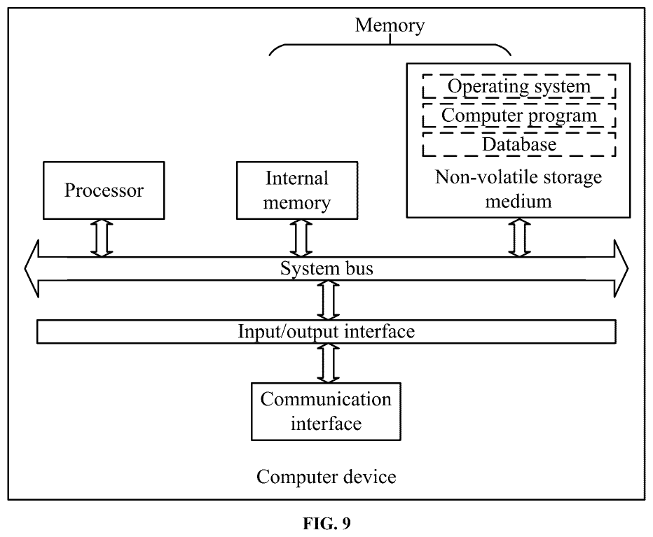

In an embodiment, a computer device is provided. The computer device may be a server or a terminal, and an internal structure thereof may be as shown in FIG. 9. The computer device includes a processor, a memory, an input/output (I/O) interface and a communication interface. The processor, the memory, and the I/O interface are connected through a system bus. The communication interface is connected to the system bus through the I/O interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for operation of the operating system and the computer program in the non-volatile storage medium. The database of the computer device is configured to store quantitative determination data of water content in concrete. The I/O interface of the computer device is configured to exchange information between the processor and an external device. The communication interface of the computer device is configured to communicate with an external terminal through a network. When executed by the processor, the computer program implements the method for quantitatively detecting water content in concrete.

Those skilled in the art may understand that the structure shown in FIG. 9 is only a block diagram of a part of the structure related to the solutions of the present application and does not constitute a limitation on a computer device to which the solutions of the present application are applied. Specifically, the computer device may include more or less components than those shown in the figure, or combine some components, or have different component arrangements.

In an embodiment, a computer device is further provided, including a memory and a processor, where the memory stores a computer program, and the computer program is executed by the processor to implement the steps of the above method embodiment.

In an embodiment, a computer-readable storage medium is provided. The computer-readable storage medium stores a computer program, and the computer program is executed by a processor to implement the steps of the above method embodiment.

In an embodiment, a computer program product is provided. The computer program product includes a computer program, and the computer program is executed by a processor to implement the steps of the above method embodiment.

It should be noted that user information (including but not limited to user device information, user personal information and the like) and data (including but not limited to data for analysis, data for storage, data for exhibition and the like) in the present application are information and data authorized by the user or fully authorized by each party, and relevant data should be acquired, used and processed according to relevant regulations.

Those of ordinary skill in the art may understand that all or some of the procedures in the method of the foregoing embodiments may be implemented by a computer program instructing related hardware. The computer program may be stored in a non-volatile computer-readable storage medium. When the computer program is executed, the procedures in the embodiments of the foregoing method may be performed. Any reference to a memory, a storage, a database, or other media used in the embodiments of the present application may include at least one of a non-volatile memory and a volatile memory. The non-volatile memory may include a read-only memory (ROM), a magnetic tape, a floppy disk, a flash memory, an optical memory, a high-density embedded nonvolatile memory, a resistive random access memory (ReRAM), a magneto-resistive random access memory (MRAM), a ferroelectric random access memory (FRAM), a phase change memory (PCM), a graphene memory, and the like. The volatile memory may include a random access memory (RAM) or an external cache memory. As an illustration rather than a limitation, the RAM may be in various forms, such as a static random access memory (SRAM) or a dynamic random access memory (DRAM).

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of the present application. The description of the foregoing embodiments is used to help illustrate the method of the present application and the core principles thereof. In addition, those of ordinary skill in the art can make various modifications in terms of specific implementations and scope of the present application in accordance with the teachings of the present application. In conclusion, the content of the specification shall not be construed as a limitation to the present application.

What is claimed is:

1. A method for quantitatively detecting water content in concrete, wherein the method for quantitatively detecting water content in concrete comprises the following steps:
obtaining an X-ray image of a to-be-detected sample, wherein the to-be-detected sample is a to-be-detected concrete sample, and the X-ray image is an image obtained after an X-ray travels through the to-be-detected concrete sample;
determining X-ray light field distribution information of the X-ray image according to the X-ray image;
determining a water density of the to-be-detected sample according to the X-ray light field distribution information; and
determining a water content of the to-be-detected sample according to the water density.

2. The method for quantitatively detecting water content in concrete according to claim 1, wherein the obtaining an X-ray image of a to-be-detected sample specifically comprises:
detecting, by an X-ray detection system, the to-be-detected sample to obtain the X-ray image, wherein
the X-ray detection system comprises an X-ray emitting apparatus, an X-ray detector, and an image processing apparatus, wherein both the X-ray emitting apparatus and the X-ray detector are connected to the image processing apparatus, the X-ray emitting apparatus and the X-ray detector are disposed opposite to each other, and the to-be-detected sample is placed between the X-ray emitting apparatus and the X-ray detector.

3. The method for quantitatively detecting water content in concrete according to claim 1, wherein the determining a water density of the to-be-detected sample according to the X-ray light field distribution information specifically comprises:
calibrating the to-be-detected sample to obtain a calibration result, wherein the calibration result comprises an attenuation coefficient curve formula and a parameter value thereof, and a water absorption function formula and a parameter value thereof; and
determining the water density of the to-be-detected sample by substituting the X-ray light field distribution information into the attenuation coefficient curve formula.

4. The method for quantitatively detecting water content in concrete according to claim 3, wherein the determining a water content of the to-be-detected sample according to the water density specifically comprises:
substituting the water density into the water absorption function formula to obtain the water content of the to-be-detected sample through calculation.

5. The method for quantitatively detecting water content in concrete according to claim 4, wherein the calibrating the to-be-detected sample to obtain a calibration result specifically comprises:
obtaining basic information of the to-be-detected sample, wherein the basic information comprises a density, a cement-to-water ratio, a cement type, a cement-to-sand ratio, gradation, and an admixture type and proportion;
preparing a calibration model with a composition the same as that of the to-be-detected sample according to the basic information of the to-be-detected sample, wherein the calibration model comprises a first calibration model and a second calibration model, and the first calibration model is the same as the second calibration model;
performing vacuum water-saturation on the first calibration model, drying the first calibration model to constant weight, soaking the second calibration model in deionized water until the second calibration model is completely saturated, separately weighing the first calibration model and the second calibration model, and determining mass of the first calibration model and mass of the second calibration model as well as a mass difference between the first calibration model and the second calibration model;
fitting a substrate surface of the first calibration model to a substrate surface of the second calibration model, and setting a side surface of the first calibration model and a side surface of the second calibration model to be perpendicular to each other to obtain an integrally spliced model;
obtaining an X-ray image of the integrally spliced model, and determining X-ray light field distribution information at each position of the integrally spliced model according to the X-ray image; and
determining each parameter value of the attenuation coefficient curve formula and each parameter value of the water absorption function formula according to the X-ray light field distribution information at each position of the integrally spliced model.

6. The method for quantitatively detecting water content in concrete according to claim 5, wherein the attenuation coefficient curve formula is as follows:
$$- ln ( \frac{I ( a , b )}{{I}_{0}} ) = \frac{{( {k}_{1} a + {k}_{2} b )}^{2} + {k}_{3} a + {k}_{4} b + {k}_{5}}{{k}_{6} a + {k}_{7} b + {k}_{8}}$$
wherein, I₀ represents an initial X-ray intensity, I(a, b) represents the X-ray light field distribution information, a represents a distance that the X-ray passes through the water-free first calibration model, b represents a distance that the X-ray passes through the watery second calibration model, and k₁, k₂, k₃, k₄, k₅, k₆, k₇, and k₈ are all parameters to be solved.

7. The method for quantitatively detecting water content in concrete according to claim 6, wherein the water absorption function formula is represented as follows:
$$Δ m = \sum \frac{Δ wb}{a + b} , and$$
$$Δ w = \frac{{m}_{2} - {m}_{1}}{{m}_{1}} ,$$
wherein, Δm represents the water content, Δw represents a water absorption rate, m₁ represents the mass of the first calibration model, m₂ represents the mass of the second calibration model, a represents the distance that the X-ray passes through the water-free first calibration model, and b represents the distance that the X-ray passes through the watery second calibration model.

8. A computer device, comprising a memory, a processor, and a computer program stored in the memory and executable on the processor, wherein the computer program is executed by the processor to implement the method for quantitatively detecting water content in concrete according to claim 1.

9. A non-transitory computer-readable storage medium, that stores a computer program thereon, wherein the computer program, when executed by a processor, implements the method for quantitatively detecting water content in concrete according to claim 1.

10. The computer device according to claim 8, wherein the obtaining an X-ray image of a to-be-detected sample specifically comprises:
detecting, by an X-ray detection system, the to-be-detected sample to obtain the X-ray image, wherein
the X-ray detection system comprises an X-ray emitting apparatus, an X-ray detector, and an image processing apparatus, wherein both the X-ray emitting apparatus and the X-ray detector are connected to the image processing apparatus, the X-ray emitting apparatus and the X-ray detector are disposed opposite to each other, and the to-be-detected sample is placed between the X-ray emitting apparatus and the X-ray detector.

11. The computer device according to claim 8, wherein the determining a water density of the to-be-detected sample according to the X-ray light field distribution information specifically comprises:
calibrating the to-be-detected sample to obtain a calibration result, wherein the calibration result comprises an attenuation coefficient curve formula and a parameter value thereof, and a water absorption function formula and a parameter value thereof; and
determining the water density of the to-be-detected sample by substituting the X-ray light field distribution information into the attenuation coefficient curve formula.

12. The computer device according to claim 11, wherein the determining a water content of the to-be-detected sample according to the water density specifically comprises:
substituting the water density into the water absorption function formula to obtain the water content of the to-be-detected sample through calculation.

13. The computer device according to claim 12, wherein the calibrating the to-be-detected sample to obtain a calibration result specifically comprises:
obtaining basic information of the to-be-detected sample, wherein the basic information comprises a density, a cement-to-water ratio, a cement type, a cement-to-sand ratio, gradation, and an admixture type and proportion;
preparing a calibration model with a composition the same as that of the to-be-detected sample according to the basic information of the to-be-detected sample, wherein the calibration model comprises a first calibration model and a second calibration model, and the first calibration model is the same as the second calibration model;
performing vacuum water-saturation on the first calibration model, drying the first calibration model to constant weight, soaking the second calibration model in deionized water until the second calibration model is completely saturated, separately weighing the first calibration model and the second calibration model, and determining mass of the first calibration model and mass of the second calibration model as well as a mass difference between the first calibration model and the second calibration model;
fitting a substrate surface of the first calibration model to a substrate surface of the second calibration model, and setting a side surface of the first calibration model and a side surface of the second calibration model to be perpendicular to each other to obtain an integrally spliced model;
obtaining an X-ray image of the integrally spliced model, and determining X-ray light field distribution information at each position of the integrally spliced model according to the X-ray image; and
determining each parameter value of the attenuation coefficient curve formula and each parameter value of the water absorption function formula according to the X-ray light field distribution information at each position of the integrally spliced model.

14. The computer device according to claim 13, wherein the attenuation coefficient curve formula is as follows:
$$- ln ( \frac{I ( a , b )}{{I}_{0}} ) = \frac{{( {k}_{1} a + {k}_{2} b )}^{2} + {k}_{3} a + {k}_{4} b + {k}_{5}}{{k}_{6} a + {k}_{7} b + {k}_{8}}$$
wherein, I₀ represents an initial X-ray intensity, I(a, b) represents the X-ray light field distribution information, a represents a distance that the X-ray passes through the water-free first calibration model, b represents a distance that the X-ray passes through the watery second calibration model, and k₁, k₂, k₃, k₄, k₅, k₆, k₇, and k₈ are all parameters to be solved.

15. The computer device according to claim 14, wherein the water absorption function formula is represented as follows:
$$Δ m = \sum \frac{Δ wb}{a + b} , and$$
$$Δ w = \frac{{m}_{2} - {m}_{1}}{{m}_{1}} ,$$
wherein, Δm represents the water content, Δw represents a water absorption rate, m₁ represents the mass of the first calibration model, m₂ represents the mass of the second calibration model, a represents the distance that the X-ray passes through the water-free first calibration model, and b represents the distance that the X-ray passes through the watery second calibration model.

16. The non-transitory computer-readable storage medium according to claim 9, wherein the obtaining an X-ray image of a to-be-detected sample specifically comprises:
detecting, by an X-ray detection system, the to-be-detected sample to obtain the X-ray image, wherein
the X-ray detection system comprises an X-ray emitting apparatus, an X-ray detector, and an image processing apparatus, wherein both the X-ray emitting apparatus and the X-ray detector are connected to the image processing apparatus, the X-ray emitting apparatus and the X-ray detector are disposed opposite to each other, and the to-be-detected sample is placed between the X-ray emitting apparatus and the X-ray detector.

17. The non-transitory computer-readable storage medium according to claim 9, wherein the determining a water density of the to-be-detected sample according to the X-ray light field distribution information specifically comprises:
calibrating the to-be-detected sample to obtain a calibration result, wherein the calibration result comprises an attenuation coefficient curve formula and a parameter value thereof, and a water absorption function formula and a parameter value thereof; and
determining the water density of the to-be-detected sample by substituting the X-ray light field distribution information into the attenuation coefficient curve formula.

18. The non-transitory computer-readable storage medium according to claim 17, wherein the determining a water content of the to-be-detected sample according to the water density specifically comprises:
substituting the water density into the water absorption function formula to obtain the water content of the to-be-detected sample through calculation.

19. The non-transitory computer-readable storage medium according to claim 18, wherein the calibrating the to-be-detected sample to obtain a calibration result specifically comprises:
obtaining basic information of the to-be-detected sample, wherein the basic information comprises a density, a cement-to-water ratio, a cement type, a cement-to-sand ratio, gradation, and an admixture type and proportion;
preparing a calibration model with a composition the same as that of the to-be-detected sample according to the basic information of the to-be-detected sample, wherein the calibration model comprises a first calibration model and a second calibration model, and the first calibration model is the same as the second calibration model;
performing vacuum water-saturation on the first calibration model, drying the first calibration model to constant weight, soaking the second calibration model in deionized water until the second calibration model is completely saturated, separately weighing the first calibration model and the second calibration model, and determining mass of the first calibration model and mass of the second calibration model as well as a mass difference between the first calibration model and the second calibration model;
fitting a substrate surface of the first calibration model to a substrate surface of the second calibration model, and setting a side surface of the first calibration model and a side surface of the second calibration model to be perpendicular to each other to obtain an integrally spliced model;
obtaining an X-ray image of the integrally spliced model, and determining X-ray light field distribution information at each position of the integrally spliced model according to the X-ray image; and
determining each parameter value of the attenuation coefficient curve formula and each parameter value of the water absorption function formula according to the X-ray light field distribution information at each position of the integrally spliced model.

20. The non-transitory computer-readable storage medium according to claim 19, wherein the attenuation coefficient curve formula is as follows:
$$- ln ( \frac{I ( a , b )}{{I}_{0}} ) = \frac{{( {k}_{1} a + {k}_{2} b )}^{2} + {k}_{3} a + {k}_{4} b + {k}_{5}}{{k}_{6} a + {k}_{7} b + {k}_{8}}$$
wherein, I₀ represents an initial X-ray intensity, I(a, b) represents the X-ray light field distribution information, a represents a distance that the X-ray passes through the water-free first calibration model, b represents a distance that the X-ray passes through the watery second calibration model, and k₁, k₂, k₃, k₄, k₅, k₆, k₇, and k₈ are all parameters to be solved.
